(19) United States
(12) Patent Application Publication
(10) Pub. No.: US 2008/0241117 A1
(43) Pub. Date: Oct. 2, 2008

(54) Oral Care Compositions Containing a Mixed Tocopherol Component

(76) Inventors: Abdul Gaffar, Princeton, NJ (US); Sarita V. Mello, Somerset, NJ (US); Michael Prencipe, Princeton Junction, NJ (US)

Correspondence Address: COLGATE-PALMOLIVE COMPANY, 909 RIVER ROAD, PISCATAWAY, NJ, 08855, US

(21) Appl. No.: 11/695,129

(22) Filed: Apr. 2, 2007

(51) Int. Cl. A61K 31/355

(52) U.S. Cl. 424/94.1; 424/673; 514/458

(57) ABSTRACT

An oral care composition, such as a dentifrice composition, which provides enhanced anti-gingivitis efficacy is disclosed. The composition includes a tocopherol component which consists of about 10% to about 90% of gamrnma tocopherol, with the balance of the components selected from alpha tocopherol, beta tocopherol, delta tocopherol, and mixtures thereof.

### BACKGROUND OF THE INVENTION

Gum disease is a form of inflammation that occurs in tissues of the oral cavity. Gingivitis, an early phase of gum disease, is an inflammation of the gums caused by the accumulation of plaque, a soft, sticky, colorless film of bacteria above the gum line. If not routinely removed by proper brushing and flossing, plaque can build up on teeth and gums and lead to gingivitis. Classic signs of gingivitis include red, swollen and tender gums that may bleed when the teeth are brushed. If not treated, gingivitis can progress to more serious gum diseases such as periodontitis and eventually to the destruction of bone and to tooth loss.

Nearly 80% of American adults have some form of gum disease. While gum disease can be treated and minimized generally with a fairly straightforward dental hygiene regimen, including regular brushing, flossing and professional dental cleanings, this hygiene routine is not always strictly followed, accounting for the relatively high presence of gum disease in the adult population. Thus, if it is possible to enhance the anti-gingivitis activity of a dentifrice, such that the routine brushing of teeth would help treat and minimize the occurrence of gum disease, that would be desirable.

Vitamin E (tocopherol) is often used in skin creams and lotions; it is reported to play a role in encouraging skin healing and reducing scarring after injuries, such as burns. Natural vitamin E exists in eight different forms or isomers, four tocopherols (alpha, beta, gamma, delta) and four tocotrienols. Some attempts to incorporate a tocopherol in oral treatments have been studied, but up until now, the results on its effect on gingival tissue are, at best, equivocal. It has been reported that the use of vitamin E resulted in a reduced level of prostaglandin E2 in gingival crevicular fluid when applied topically in rinse form. Several investigators have evaluated the use of vitamin E in the form of alpha tocopherol for its effect on gingivitis and periodontitis. It has been reported that alpha tocopherol, when applied topically in toothpaste, penetrates gum tissue, but fails to provide an effect on gingivitis in primates and humans.

Vitamin E has been studied for many health effects other than periodontal disease. For example, Violi et al., Ann. NY Acad. Sci. 1031:292-304 (2004), analyzes the literature to determine whether it supports the premise that vitamin E has a positive effect on the treatment of cardiovascular disease. Additionally, Panganamala et al., Prostaglandins, 14(2): 261-271 (1977), describes use of tocopherol to inhibit arachidonic acid-induced platelet aggregation and ADP induced platelet aggregation, as well as the activity of soybean lipoxidase.

### BRIEF SUMMARY OF THE INVENTION

The invention includes an oral care composition that comprises about 0.1% to about 5% of a tocopherol component. The tocopherol component consists of about 50% to about 90% by weight of gamma tocopherol and the balance of the tocopherol component is selected from alpha tocopherol, beta tocopherol, delta tocopherol, and mixtures thereof. The invention also includes a method of improving or maintaining the systemic health of a mammal that comprises applying to an oral surface of the mammal a composition comprising about 0.1% to about 5% of a tocopherol component. The tocopherol component consists of at least about 50% to about 90% by weight of gamma tocopherol and the balance of the tocopherol component is selected from alpha tocopherol, beta tocopherol, delta tocopherol, and mixtures thereof.

### BRIEF DESCRIPTION OF THE DRAWINGS

FIG. 1 illustrates average pocket depth at baseline and at one mark for Toothpaste A and Toothpaste B (bars are mean values, whiskers are standard errors).

FIG. 2 illustrates plaque prevalence at baseline and at one month for Toothpaste A and Toothpaste B (bars are mean values and whiskers are standard errors).

### DETAILED DESCRIPTION OF THE INVENTION

The present invention relates to dentifrice compositions and other oral care compositions containing mixed tocopherol materials, which provide improved anti-gingivitis efficacy.

The present invention relates to oral care compositions, such as dentifrices, toothpastes, tooth powders, or oral rinses. These compositions when applied orally may provide the user with an anti-gingivitis benefit. The present invention, comprises a mixed tocopherol component, together with conventional components found in oral care/dentifrice compositions.

Tocopherol, or vitamin E, is a fat-soluble vitamin that exists in eight different forms or isomers, four tocopherols and four tocotrienols. There is an alpha, beta, gamma and delta form of both the tocopherols and the tocotrienols, determined by the number of methyl groups on the chromanol ring. Each form has its own biological activity. The tocopherol forms are represented by the structure:

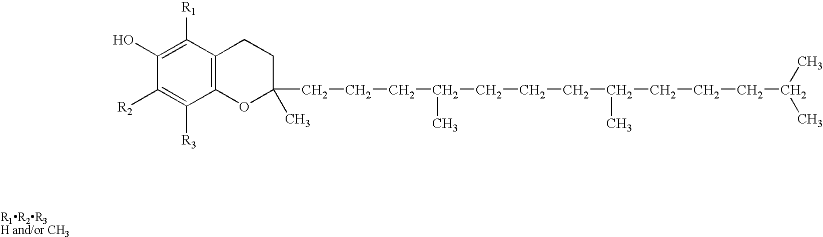

wherein each of R₁, R₂ and R₃, is a hydrogen atom or a −CH₃, depending on the form, as shown in Table I.

TABLE I

| | R₁ | R₂ | R₃ |
|---|---|---|---|
| Alpha-TOCOPHEROL | CH₃ | CH₃ | CH₃ |
| Beta-TOCOPHEROL | CH₃ | H | CH₃ |
| Gamma-TOCOPHEROL | H | CH₃ | CH₃ |
| Delta-TOCOPHEROL | H | H | CH₃ |

The tocopherol component used in the invention may contain tocopherol obtained from any sources, natural or synthetic. Conventional sources include vegetable oils, whole grains, fish, nuts, sea buckthorn, and leafy green vegetables.

The tocopherol component utilized in the compositions of the present invention consists of about 10% to about 90% or about 50% to about 90% (of the tocopherol component) of gamma tocopherol, with the balance of the component being selected from alpha tocopherol, beta tocopherol, delta tocopherol and mixtures of those materials. In one embodiment, the tocopherol component consists of about 50% to about 90% gamma tocopherol, with the balance of the component being selected from alpha tocopherol, beta tocopherol, and mixtures of those materials. In another embodiment, the tocopherol component includes about 50% to about 90% gamma tocopherol or about 10% to about 90% gamma tocopherol, with the balance of the component being alpha tocopherol. In yet another embodiment of the present invention, the tocopherol component includes a 50:50 mixture of garnma tocopherol and alpha tocopherol. Other combinations, falling within the definition given above, may also be used. The tocopherol component is generally present in the oral care compositions of the present invention at about 0.1% to about 5%, such as about 0.5 to about 1.5% of the oral care (e.g., dentifrice) compositions.

The tocopherol used in the invention may be obtained by any means known or to be developed in the art. Methods of synthesizing tocopherol, as well as the chromatographic techniques for separating out the various isomers of tocopherol are well known in the art. See, for example Lienau, et al., Analytical Chemistry, 74(20): 5192-5198 (2002).

Any conventional oral care vehicles used, for example, in dentifrices, tooth powders and oral rinses can be used in the present invention. Vehicles used to prepare the dentifrice compositions of the present invention comprise a water phase containing a humectant therein. The humectant may be, for example, glycerin, sorbitol, xylitol, and/or propylene glycol of molecular weight in the range of 200-1000, but other humectants and mixtures thereof may also be employed. The humectant concentration may constitute about 5% to about 70% by weight of the oral composition.

The dentifrice compositions of the present invention can contain a variety of optional dentifrice ingredients. As described below, such optional ingredients can include, but are not limited to, thickening agents, surfactants, a source of fluoride ions, a synthetic anionic polycarboxylate, a flavoring agent, abrasives, additional anti-plaque agents and coloring agents. Other agents that may be included are stannous ion agent, triclosan, triclosan monophosphate, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, delmopinol, octaphinol, nisin, zinc ion agent, copper ion agent, essential oils, furanones, bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, propolis, and arginine (free base or salt).

Thickeners, used in the compositions of the present invention may include natural and synthetic gums and colloids, examples of which include carrageenan (rich moss), xanthan gum and sodium carboxymethyl cellulose, starch, polyvinyl pyrrolidone, hydroxyethyl propyl cellulose, hydroxybutyl methyl cellulose, hydroxypropyl methyl cellulose, and hydroxyl ethyl cellulose. Inorganic thickeners include amorphous silica compounds which function as thickening agents and include colloidal silica compounds available under trademarks including Cab-o-Sil, fumed silica manufactured by Cabot Corporation and distributed by Lenape Chemical, Bound Brook, N.J.; Zeodent 165 from J. M. Huber Chemicals Division, Havre deGrace, Md.; and Sylox 15, also known as Sylodent 15, available from Davidson Chemical Division of W. R. Grace Corporation, Baltimore, Md. The thickening agent is generally present in the dentifrice composition in an amount of about 0.1% to about 10% by weight, more specifically about 0.5% to about 40% by weight of the composition.

Surfactants may be used in the compositions of the present invention to achieve increased prophylactic action and render the dentifrice compositions more cosmetically acceptable. The surfactant is preferably a detersive material which imparts to the composition detersive and foaming properties. Surfactants are frequently anionic, although other surfactants such as nonionic surfactants, can also be used. Suitable examples of surfactants are water-soluble salts of higher fatty acid monoglyceride monosulfates, such as the sodium salt of monosulfated monoglyceride of hydrogenated coconut oil fatty acids, higher alkyl sulfates, such as sodium lauryl sulfate, alkyl aryl sulfonates, such as sodium dodecyl benzene sulfonate, higher alkyl sulfoacetates, such as sodium lauryl sulfoacetate, higher fatty acid esters of 1,2-dihydroxypropane sulfonate, and the substantially saturated higher aliphatic acyl amides of lower aliphatic amino carboxylic compounds, such as those having 12-16 carbons in the fatty acid, alkyl or acyl radicals, and the like. Examples of the last mentioned amides are N-lauryl sarcosine, and the sodium, potassium and ethanolamine salts of N-lauryl, N-myristoyl, or N-palmitoyl sarcosine. This surfactant is typically present in the dentifrice compositions of the present invention in an amount of about 0.3% to about 5% by weight, more specifically about 0.5% to about 2% by weight.

Nonionic surfactants may also be utilized in the dentifrice compositions of the present invention. Those materials include nonanionic polyoxyethylene surfactants, such as Polyoxamer 407, Steareth 30, Polysorbate 20, and PEG-40 Castor Oil, and amphoteric surfactants, such as cocamidopropyl betaine (tegobaine), and cocamidopropyl betaine lauryl glucoside, condensation products of ethylene oxide with various hydrogen containing compounds that are reactive therewith and have long hydrocarbon chains (e.g., aliphatic chains of about 12 to about 20 carbon atoms), which condensation products (ethoxamers) contain hydrophilic polyoxyethylene moieties, such as condensation products of poly (ethylene oxide) with fatty acids, fatty alcohols, fatty amides and other fatty moieties, and with propylene oxide and polypropylene oxides (e.g., Pluronic™ materials).

The dentifrice composition of the present invention may also contain a source of fluoride ions or a fluoride-providing component, as an anticaries agent in an amount sufficient to supply about 25 ppm to about 5,000 ppm of fluoride ions and include inorganic fluoride salts, such as soluble alkaline metal salts, for example, sodium fluoride, potassium fluoride, sodium fluorosilicate, ammonium fluorosilicate, sodium monofluorophosphate, as well as tin fluorides, such as stannous fluoride and stannous chloride. Sodium fluoride is a compound used in particular embodiments of the present invention.

In addition to fluoride compounds, there may also be included anti-tartar agents such as pyrophosphate salts including dialkali or tetraalkali metal pyrophosphate salts, such as Na₄P₂O₇, K₄P₂O₇, Na₂K₂P₂O₇, Na₂H₂P₂O₇, and K₂H₂P₂O₇, long-chain polyphosphates such as sodium hexametaphosphate, and cyclic phosphates such as sodium trimetaphosphate. These anticaries agents are included in the dentifrice compositions in a concentration of about 1% to about 5% by weight.

Another active agent useful in the dentifrice compositions of the present invention is an antibacterial agent, which can be present at about 0.2% to about 1% by weight of the dentifrice composition. Such useful antibacterial agents include non-cationic antibacterial agents which are based on phenolic or bisphenolic compounds, such as halogenated diphenyl ethers, such as triclosan (2,4,4′-trichloro-2′-hydroxydiphenyl ether).

Synthetic anionic polycarboxylates may also be used in the dentifrice compositions of the present invention as an efficacy enhancing agent for any antibacterial, anti-tartar or any other active agent within the dentifrice composition. Such anionic polycarboxylates are generally employed in the form of their free acids or, preferably, partially or more preferably fully neutralized water soluble alkali metal (for example, potassium and sodium) or ammonium salts. One embodiment of the present invention includes 1:4:1 copolymers of maleic anhydride or acid with another polymerizable ethylenically unsaturated monomer, preferably methyl vinyl esther maleic anhydride having a molecular weight (MW) of about 30,000 to 1,800,000, more specifically about 30,000 to 700,000. Examples of these copolymers are available from GAF Corporation under the trade name Gantrez, for example, AN139 (MW=500,000), AN119 (MW=250,000); S-97 pharmaceutical grade (MW=700,000), AN169 (MW=1,200,000-1,800,000), and AN179 (MW=above 1,800,000); wherein a specific polymer which can be utilized in the present invention is S97 pharmaceutical grade (MW=700,000).

When present, the anionic polycarboxylate is employed in amounts effective to achieve the desired enhancement of the efficacy of any antibacterial, antitartar, or other active agent within the dentifrice composition. Generally, the anionic polycarboxylate is present within the dentifrice composition at about 0.05% to about 4% by weight, more specifically, about 0.5% to about 2.5% by weight, of the composition.

The dentifrice compositions of the present invention may include abrasives, such as precipitated silicas having a mean particle size of up to about 20 microns, such as Zeodent 115, marketed by J. M. Huber Chemical Division, Havre de Grace, Md., or Silodent, 783, marketed by Davison Chemical Division of W. R. Grace and Company. Other useful dentifrice abrasives include metaphosphate, potassium metaphosphate, tricalcium phosphate, dihydrated dicalcium phosphate, aluminum silicate, calcined alumina, bentonite, or other silaceous materials, or combinations thereof. Specific embodiments of abrasive materials useful in the present invention include silica gels and precipitated amorphous silicas having an oil absorption value of less than about 100 cc/100 g silica and more specifically in the range of about 45 cc/100 g to less than about 70 cc/100 g silica. These silicas are colloidal particles having an average particle size of about 3 microns to about 12 microns or about 5 microns and about 10 microns, and a pH of about 4 to about 10, or about 6 to about 9 when measured as a 5% by weight slurry.

Oil absorption values are measured using the ASTM Rub-Out Method D281. The low oil absorption silica abrasives are present in the oral composition of the present invention, when used, at a concentration of about 5% to about 40% by weight, alternatively of about 10% to about 30% by weight.

Examples of low absorption silica abrasives useful in the present invention are marketed under the trade designation Sylodent XWA by Davison Chemical, a division of W. R. Grace and Company, Baltimore, Md.; and Sylodent 650 XWA, a silica hydrogel, composed of particles of colloidal silica, having a water content of 29% by weight. The particles may, for example, be about 7 to about 10 microns in diameter, and have an oil absorption of less than 70 cc/100 g of silica.

The silica used in the compositions of the invention may have varying abrasivity. However, it may be desirable that the silica has a pellicle cleaning ration (PCR) value of greater than about 90 and a radioactive dentin abrasion (RDA) value of less than about 250.

The dentifrice composition of the present invention may also contain a flavoring agent. Flavoring agents which are used in the practice of the present invention include essential oils, as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint and spearmint. The flavoring agent is incorporated in the dentifrice composition at a concentration of about 0.1% to about 5% by weight, more specifically of about 0.5% to about 1.5% by weight.

Various other materials may be incorporated in dentifrice compositions of the present invention, including desensitizers, such as potassium nitrate; whitening agents, such as hydrogen peroxide, calcium peroxide, and urea peroxide; preservatives; silicones; pigments/colorants; and chlorophyll compounds. These additives, when present, are incorporated in the dentifrice composition in their conventional amounts and specifically in amounts which provide their desired benefits but do not substantially adversely affect the properties and characteristics desired for the dentifrice composition itself.

The preparation of a dentifrice is well known in the art, and is described, for example, in U.S. Pat. Nos. 3,966,863; 3,980,767; 4,328,205; and 4,358,437, all of which are incorporated herein by reference. More specifically, to prepare a dentifrice of the present invention, generally, the humectant (e.g., glycerin, sorbitol, propylene glycol, and/or polyethylene glycol) is dispersed in water in a conventional mixer under agitation. Into that dispersion are added the organic thickeners, such as carboxyl methyl cellulose (CMC), carrageenan, or xanthan gum; any anionic polycarboxylate; any salts, such as sodium fluoride anticaries agents; and any sweeteners; the resultant mixture is agitated until a homogeneous gel phase is formed. Into the gel phase are added any pigments utilized, such as TiO₂, and any acid or base required to adjust the pH of the composition. These ingredients are mixed until a homogeneous phase is obtained. The mixture is then transferred to a high speed/vacuum mixer, wherein the inorganic thickener, such as Zeodent 165, and the surfactant ingredients are added to the mixture. Any abrasives utilized are added at this point. Any water insoluble bacterial agents, such as triclosan, are solublized in the flavor oils to be included in the dentifrice and that solution is added along with the surfactants to the mixture, which is then mixed at high speed for about five to about 30 minutes, under a vacuum of about 20 to about 50 mm of Hg, specifically about 30 mm Hg. The resultant product is a homogeneous, semi-solid, extrudable paste or gel product.

### EXAMPLE I

Using the preparation method described above, the following four compositions of the present invention are prepared. The mixed tocopherols noted in Table II comprise a 50:50 mixture of gamma and alpha tocopherols.

TABLE II

| Raw Material | Formula 1 | Formula 2 |
|---|---|---|
| Sodium Fluoride | 0.243 | 0.243 |
| Polyethylene Glycol | 3 | 3 |
| Propylene Glycol | — | 0.5 |
| Tocepherols | 1.0 | 1.0 |
| Sodium CMC | 0.6 | 0.6 |
| Sorbitol | 59.7 | 60 |
| Sodium Saccharin | 0.3 | 0.3 |
| Tetrasodium Pyrophosphate | 0.5 | 2 |
| Zeodent 115-Abrasive | 25.5 | 25.5 |
| Sylodent XW A650-High | — | 10 |
| Flavor | 0.72 | 1 |
| Sodium Lauryl Sulfate | 1.5 | 1.5 |
| Water | Balance | Balance |
| pH | 6.5–8.5 | 6.5–8.5 |

The dentifrice compositions prepared, when used on a regular basis, are effective in cleaning teeth and in providing an anti-gingivitis benefit to the user.

### EXAMPLE II

Forty-one subjects of mixed gender, race, and demographics were selected. All subjects were between the ages of 18 to 65, in good general health, and had been diagnosed with gingivitis of varying severity.

The subjects were separated into two groups: A and B.

Group A was instructed to brush twice daily with a toothpaste of Formulation A (See Table III). (“Toothpaste A”)

Group B was instructed to brush twice daily with a toothpaste of Formulation B (See Table III). (“Toothpaste B”)

TABLE III

| Material | Formula A | Formula B |
|---|---|---|
| Sodium fluoride | 0.24 | 0.24 |
| Saccharin | 0.30 | 0.30 |
| Sorbitol | 59.00 | 60.00 |
| Polyethylene glycol | 3.00 | 3.00 |
| Carboxymethyl cellulose | 0.60 | 0.60 |
| Tetrasodium pyrophosphate | 0.50 | 0.50 |
| Silica abrasive | 25.50 | 25.50 |
| Flavor | 0.72 | 0.72 |
| Surfactant | 1.50 | 1.50 |
| Gamma tocopherol | 0.50 | 0 |
| Natural Vitamin E | 0.50 | 0 |
| Water | Balance | Balance |

The subjects were examined at baseline visit (prior to toothpaste use) and a two month visit.

During each visit, various measurements of oral health were carried out to include measurements of gingival pocket depth and plaque.

Pocket depth was measured from the free gingival margin to the base of the pocket and recorded in whole millimeters. In each subject, pocket depth was measured at six sites (mesiobuccal, buccal, distobuccal, mesiolingual, lingual, and distolingual).

Plaque was measured in each subject at 168 sites in accordance with the methods of Loe et al., The gingival index, the plaque index, and the retention index. J. Periodontal., 1967 38:610-616.

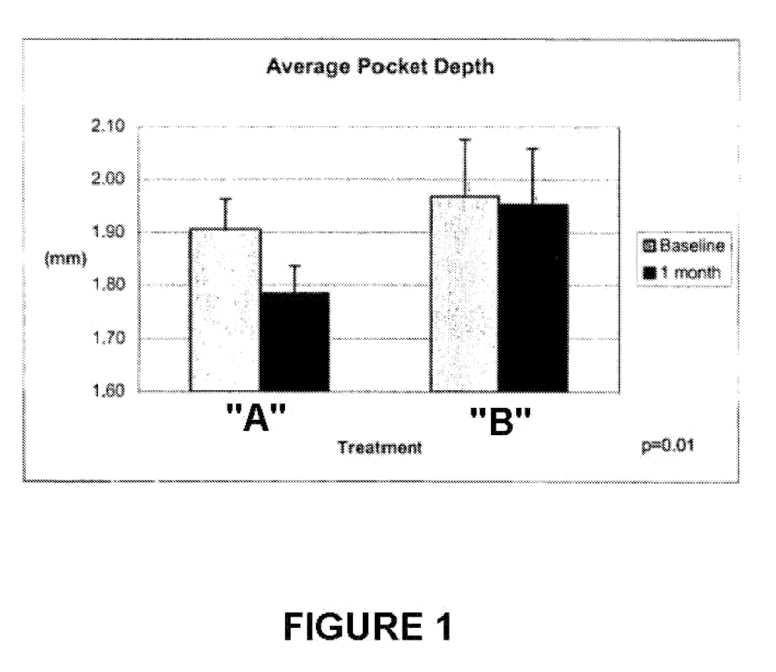

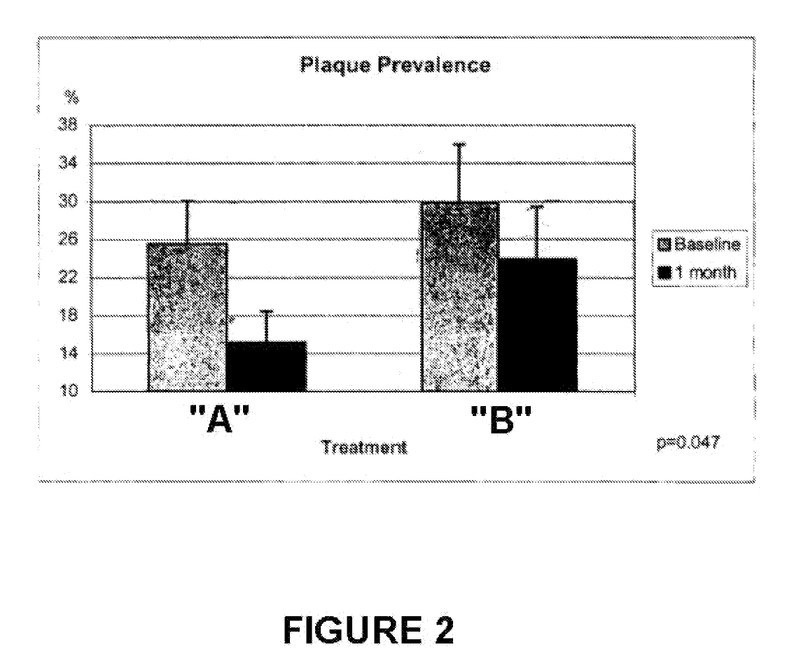

Results for each of these parameters after one month brushing with Toothpaste A or Toothpaste B are shown in FIGS. 1 and 2.

FIG. 1 shows that the average pocket depth of the subjects using Toothpaste A for one month was reduced as compared to the average pocket depth of those subjects using Toothpaste B.

FIG. 2 shows that the subjects using Toothpaste A for one month experienced greater reduction in plaque formation than those using Toothpaste B.

1. An oral care composition comprising about 0.1% to about 5% of a tocopherol component, wherein the tocopherol component consists of about 10% to about 90% by weight of gamma tocopherol and the balance of the tocopherol component is selected from alpha tocopherol, beta tocopherol, delta tocopherol, and mixtures thereof.

2. The composition of claim 1 wherein the tocopherol component is present in an amount of about 0.5% to 1.5% by weight of the composition.

3. The composition of claim 1 wherein the balance of the tocopherol component is selected from alpha tocopherol, beta tocopherol, and mixtures thereof.

4. The composition of claim 1 where in balance of the tocopherol component is alpha tocopherol.

5. The composition of claim 1 wherein the tocopherol component is a 50:50 mixture of gamma tocopherol and alpha tocopherol.

6. The composition of claim 1, further comprising a fluoride ion source.

7. The composition of claim 1 further comprising a stannous ion agent, triclosan, triclosan monophosphate, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, delmopinol, octaphinol, nisin, zinc ion agent, copper ion agent, essential oils, furanones, bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, propolis, and arginine (free base or salt).

8. The composition of claim 1 having a pH of at least about 5.

9. The composition of claim 1, wherein the composition further comprises an agent selected from an abrasive agent, an antibacterial agent, a plaque dispersion agent, an antiadhesion agent, an anticaries agent, a desensitizing agent, a flavorant, a colorant, and a sensate.

10. The composition of claim 1 further comprising about 5% to about 75% of a humectant selected from glycerin, sorbitol, propylene glycol, and mixtures thereof.

11. A method of improving or maintaining the systemic health of a mammal comprising applying to an oral surface of the mammal a composition comprising about 0.1% to about 5% of a tocopherol component, wherein the tocopherol component consists of about 10% to about 90% by weight of gamma tocopherol and the balance of the tocopherol component is selected from alpha tocopherol, beta tocopherol, delta tocopherol, and mixtures thereof.

12. The method of claim 11, wherein the composition further comprises an agent selected from a stannous ion agent, triclosan, triclosan monophosphate, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, delmopinol, octaphinol, nisin, zinc ion agent, copper ion agent, essential oils, furanones, bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, propolis, and arginine (free base or salt).

13. A method of ameliorating or/or preventing gum inflammation, gingivitis and/or periodontitis in a mammal comprising applying to and oral surface of mammal composition comprising about 0.1% to about 5% of a tocopherol component, wherein the tocopherol component consists of about 10% to about 90% by weight of gamma tocopherol and the balance of the tocopherol component is selected from alpha tocopherol, beta tocopherol, delta tocopherol, and mixtures thereof.

14. The method of claim 13, wherein the composition further comprises an agent selected from a stannous ion agent, triclosan, triclosan monophosphate, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, delmopinol, octaphinol, nisin, zinc ion agent, copper ion agent, essential oils, furanones, bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, propolis, and arginine (free base or salt).

15. The method of claim 13, wherein the tocopherol component is present in an amount of about 0.5% to 1.5% by weight of the composition.

16. The method of claim 13, wherein the balance of the tocopherol component is selected from alpha tocopherol, beta tocopherol, and mixtures thereof.

17. The method of claim 13, wherein the balance of the tocopherol component is alpha tocopherol.

18. The method of claim 17, wherein the tocopherol component is a 50:50 mixture of gamma tocopherol and alpha tocopherol.

19. An oral care composition comprising about 0.1% to about 5% of a tocopherol component, wherein the tocopherol component consists of about 10% to about 90% gamma tocopherol and the balance of the tocopherol component is alpha tocopherol.

20. A method of ameliorating and/or preventing gum inflammation, gingivitis and/or periodontitis, the method comprising contacting an oral surface with an oral care composition at least twice daily for a period of about 1 month to about 1 year, wherein the oral care composition comprises about 0.1% to about 5% of a tocopherol component, wherein the tocopherol component consists of about 10% to about 90% by weight of gamma tocopherol and the balance of the tocopherol component is selected from alpha tocopherol, beta tocopherol, delta tocopherol, and mixtures thereof wherein a ratio of the first abrasive to the second abrasive ranges of about 1:1.6 to about 1.6:1, and wherein the periodic oral composition has a pellicle cleaning ratio of greater than about 90 and a radioactive dentin abrasion of less than about 250.

21. A method for reducing or inhibiting plaque formation on an oral surface comprising applying to an oral surface of a mammal a composition comprising about 0.1% to about 5% of a tocopherol component, wherein the tocopherol component consists of about 10% to about 90% by weight of gamma tocopherol and the balance of the tocopherol component is selected from alpha tocopherol, beta tocopherol, delta tocopherol, and mixtures thereof.

22. A method of reducing the depth of a periodontal pocket comprising applying to an oral surface of a mammal a composition comprising about 0.1% to about 5% of a tocopherol component, wherein the tocopherol component consists of about 10% to about 90% by weight of gamma tocopherol and the balance of the tocopherol component is selected from alpha tocopherol, beta tocopherol, delta tocopherol, and mixtures thereof.
